(19) United States
(12) Plant Patent Application Publication
(10) Pub. No.: US 2004/0205874 P1
(43) Pub. Date: Oct. 14, 2004

(54) St. Augustine grass named 'B12'

(76) Inventor: Todd Anthony Layt, New South Wales (AU)

Correspondence Address: MYERS BIGEL SIBLEY & SAJOVEC, PO BOX 37428, RALEIGH, NC, 27627, US

(21) Appl. No.: 10/663,928

(22) Filed: Sep. 16, 2003

(30) Foreign Application Priority Data
Nov. 26, 2002 (AU) 2002/342

(51) Int. Cl. A01H 5/00

(52) U.S. Cl. PLT/392

(57) ABSTRACT

‘B12’ is an attractive, green (RHS 137A) Stenotaphrum secundatum grass (St. Augustine grass), with a fast growth habit, brown with predominant yellow-green internode color, moderate internode length, and a long leaf blade that is highly infolded in mature turf, with a fine leaf appearance. ‘B12’ is also characterized by good disease and pest resistance and superior color retention under low fertilizer conditions.

### RELATED APPLICATION INFORMATION

This application claims the benefit of Australian Plant Breeders Rights Application Serial No. 2002/342, filed on Nov. 15, 2003, received by the Australian Plant Breeders Rights Office on Nov. 26, 2002, and accepted on Dec. 13, 2003.

### LATIN NAME OF THE GENUS AND SPECIES

The Latin name of the genus and species of the novel variety disclosed herein is Stenotaphrum secundatum.

### VARIETY DENOMINATION

The inventive variety of St. Augustine grass disclosed herein has been given the variety denomination ‘B12’.

### BACKGROUND OF THE INVENTION

Stenotaphrum secundatum belongs to the grass family, Gramineae, and has the common names St. Augustine grass and buffalo grass. Stenotaphrum secundatum is a vigorous-growing perennial grass that prefers warm weather climates, and is able to withstand temperatures as high as 105° F. Its market class is turf grass, and it is widely used as a lawn grass in the Southern United States where other grasses cannot survive the extreme temperatures.

Some commonly known varieties of S. secundatum include ‘Floratine’ (unpatented), ‘Bitter Blue’ (unpatented), ‘Floratam’ (unpatented), ‘Seville St. Augustine’ (unpatented), ‘Raleigh St. Augustine’ (unpatented), ‘Texas Common’ (unpatented), ‘SS-100’ (U.S. Plant Pat. No. PP9,395; sold under the name PALMETTO™), ‘Shademaster’ (unpatented), ‘Sir Walter’ (Australian PBR No. 96/226; unpatented in the United States), and ‘ST-85’ (Australian Patent No. 643567; unpatented in the United States).

Lineage:

The variety ‘B12’ was identified in Spring, 2001 in Clarendon, New South Wales, Australia, during a seedling selection of cultivated ‘Sir Walter’. ‘Sir Walter’ is characterized by long-average internode length and intense purple internode color. Selection criteria for ‘B12’ were greener internode color and shorter internode length. The parent plant ‘Sir Walter’ was grown in isolation, and 5000 seeds were collected from open pollination in Spring 2000. These seeds were sown, and in February and March, 2001, twelve of the resulting plants were selected based on their green stems. In Spring, 2001 a final single selection, designated ‘B12’, was made from these twelve seedlings based on shorter internode length.

Asexual Reproduction:

‘B12’ was first asexually propagated by stolons in September, 2001 in Clarendon, New South Wales, Australia. ‘B12’ has since been asexually propagated by means of stolons. The distinctive characteristics of the variety have remained stable and true to type through successive cycles of asexual propagation.

### SUMMARY OF THE INVENTION

‘B12’ is an attractive, green (RHS 137A) Stenotaphrum secundatum grass (St. Augustine grass), with a fast growth habit, brown with predominant yellow-green internode color, moderate internode length, and a long leaf blade that is highly infolded in mature turf, with a fine leaf appearance. ‘B12’ is also characterized by good disease and pest resistance and superior color retention under low fertilizer conditions.

### BRIEF DESCRIPTION OF THE DRAWINGS

FIG. 1. Stolon length and color. Photo taken July 2003 in New South Wales, Australia showing the back view of the stolon. ‘Sir Walter’ with its longer internode length, and more purple internode is on the left. ‘B12’ with its medium internode length and its lighter colored internode is in the center. ‘SS-100’ is to the right with its shorter and greener internode.

FIG. 2. Stenotaphrum ‘B12’ (left) with comparator ‘SirWalter’ (center) and ‘Shademaster’ (right) showing differences in internode color.

FIG. 3. Leaf angle comparison among ‘SS-100’ (left), ‘B12’ (middle), and ‘Sir Walter’ (right).

FIG. 4. “B12’ has a finer leaf and a higher frequency of acute leaf angles. Measurements for leaf angle trial (Comparison Trial 4) came from within this ring.

FIG. 5. ‘Sir Walter’ has a less fine leaf, and a higher frequency of less acute leaf angles. Measurements for leaf angle trial (Comparison Trial 4) came from within this ring.

FIG. 6. ‘SS100’ has a less fine leaf, and a higher frequency of more open leaf angles than ‘B12’. Measurements for leaf angle trial (Comparison Trial 4) came from within this ring.

FIG. 7. ‘B12’ is shown as 18-month-old turf, illustrating its fine leaf appearance, with an Australian 50 cent piece in the picture.

FIG. 8. ‘Sir Walter’ is shown as 18-month-old turf, illustrating its broader appearing leaf as compared with ‘B12’, with an Australian 50 cent piece in the picture.

FIG. 9. ‘SS100’ is shown as 18-month-old turf, illustrating its broader appearing leaf as compared with ‘B12’, with an Australian 50 cent piece in the picture.

FIG. 10. Shows clippings collected from samples of each Stenotaphrum evaluated in the low fertilizer trials. At the end of the Kincumber trial, all vegetative material was harvested from each pot, placed in a plastic bag and weighed. (see Table 8). ‘B12’ and ‘SS-100’ grew better with no fertilizer, as compared with the other varieties.

FIG. 11. Pots of the different Stenotaphrum showing the excellent growth of the ‘B12’ and ‘SS-100’ (labeled as ‘Palmetto’) with no fertilizer. The long leaves of ‘B12’ can also be seen.

FIG. 12. PCR products separated on a 1.5% agarose gel in Tris-borate-EDTA containing ethidium bromide. PCR amplification products were visualized using a Bio-Rad transilluminator. Lane 1=‘Bitterblue’; Lane 2=‘Palmetto’™; Lane 3=‘Woerner Classic’; Lane 4=‘Raleigh St. Augustine’; Lane 5=‘Floratam’; Lane 6=‘B12’.

### DETAILED BOTANICAL DESCRIPTION OF THE VARIETY

The following is a detailed botanical description of a new Stenotaphrum secundatum grass variety known as ‘B12’, based upon observations of the plant grown in nursery pots and field plots. Those skilled in the art will appreciate that certain characteristics will vary with older or, conversely, with younger plants. ‘B12’ has not been observed under all possible environmental conditions. Where dimensions, sizes, colors and other characteristics are given, it is to be understood that such characteristics are approximations or averages set forth as accurately as practicable. The phenotype of the variety may differ from the descriptions herein with variations in the environment such as season, temperature, light intensity, day length, cultural conditions, and the like. Color notations are based on The Royal Horticultural Society Colour Chart, The Royal Horticultural Society, London (1995 edition).

‘B12’ is a perennial, vegetatively propagated Stenotaphrum grass, believed to be a variety of Stenotaphrum secundatum. The parent of ‘B12’ is ‘Sir Walter’ (Australian PBR no. 96/226; unpatented in the United States), a variety of Stenotaphrum widely grown in Australia. ‘B12’ is a fine leafed, fastgrowing variety.

### TECHNICAL DESCRIPTION OF THE VARIETY

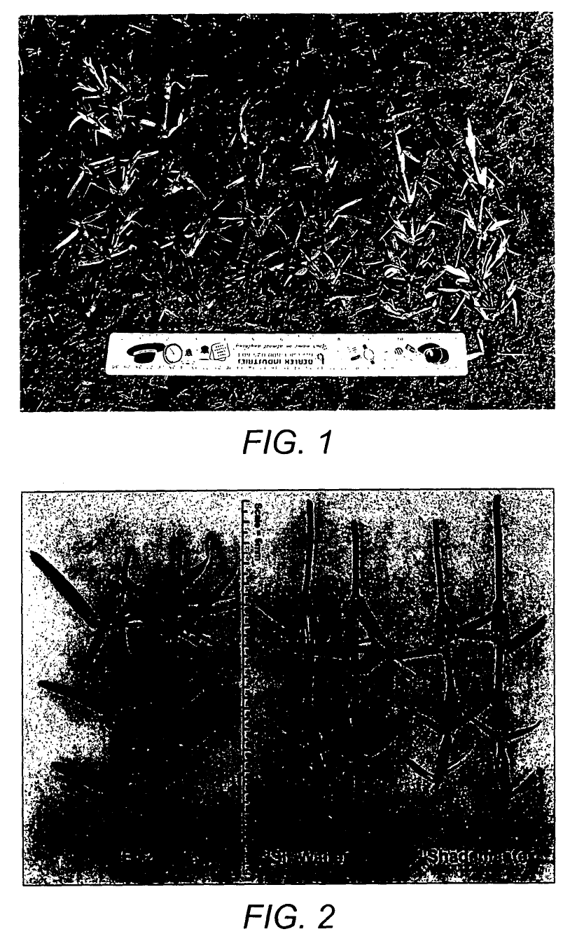

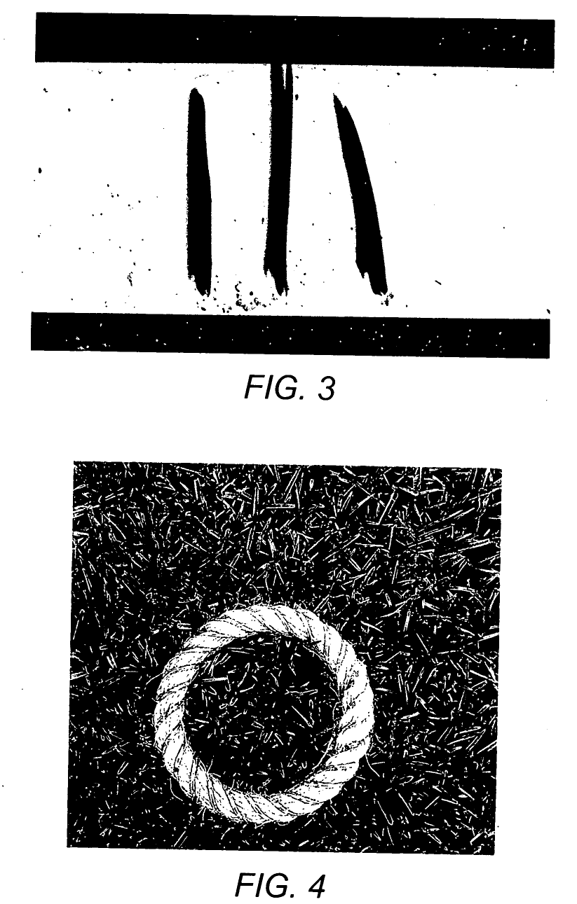

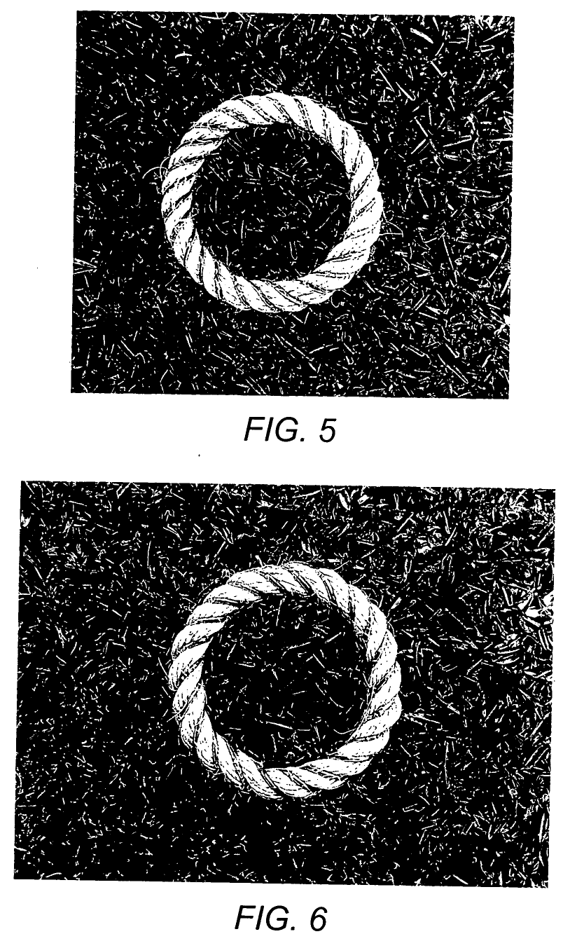

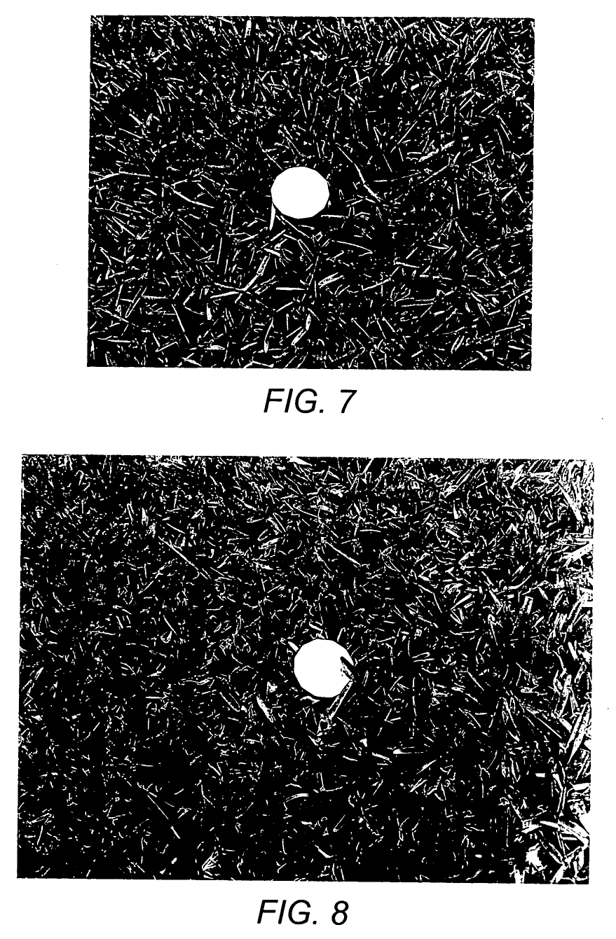

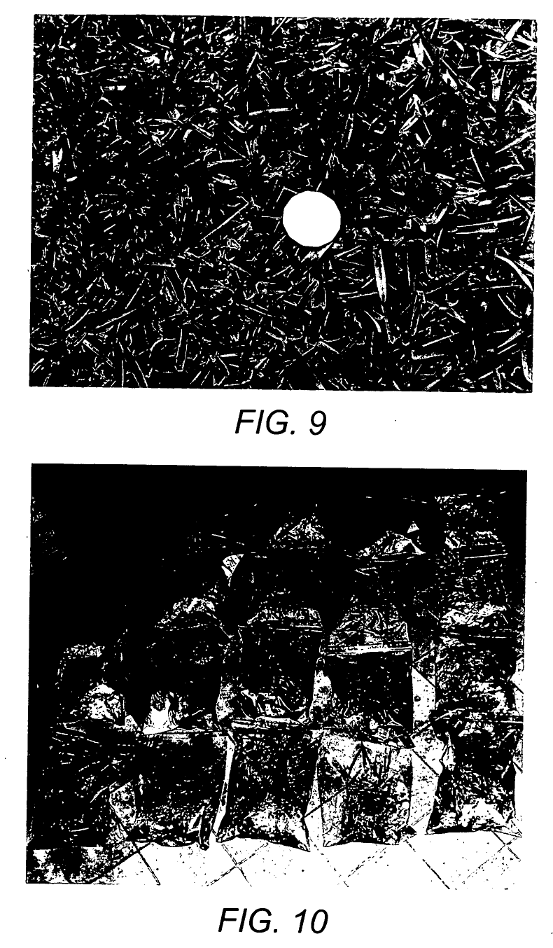

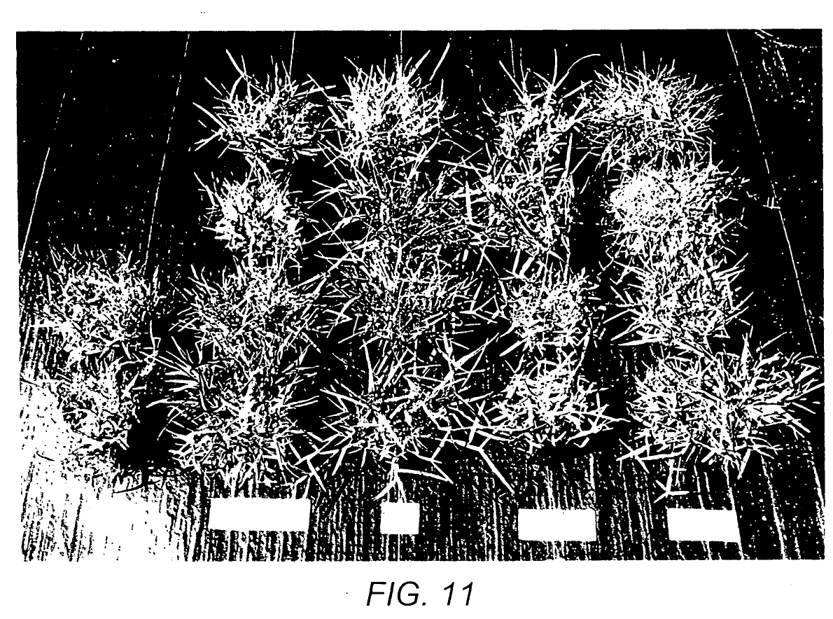

The description of the variety below is taken from a pot trial conducted in the Spring/Summer 2002 in Clarendon, New South Wales, Australia. (Comparison Trial 1 below). Additional characteristics of the plant are illustrated in FIGS. 1-11.

Plant characteristics: Perennial, stoloniferous grass, habit prostrate becoming erect when flowering, culms branched, glabrous.

Stolon: Roots at nodes, internode length (4th from tip) medium-long (mean 48.6 mm), average internode length (internodes 4 to 6 from tip) medium-long (mean 50.4 mm), color yellow green (RHS 144A) at node changing to yellow green (RHS 148A) along internode diffuse with brown (RHS 200A) becoming predominantly brown (RHS 200A) on upper exposed side of internode with maturity.

Leaf: Sheath length medium (mean 19.1 mm), color green (RHS 138B), blade length medium (mean 20.2 mm), blade width medium (mean 6.4 mm), color green (RHS 137A), apex acute.

Inflorescence: Spike-like panicle.

Cultural notes: ‘B12’ has been observed to survive to a temperature of —10° Celsius, and is heat and humidity tolerant. It has good drought tolerance, comparable with ‘Sir Walter’, and ‘SS-100’ (sold under the name PALMETTO™; U.S. Plant Pat. No. PP9,395), with a strong and vigorous root system. ‘B12’ also grows well under low fertilizer conditions. Shade tolerance is good and is being further evaluated. ‘B12’ competes well with invasive weeds. It strikes well when being stolonised, and transplants well as sod.

Disease resistance: ‘B12’ appears to be resistant to grey-leaf spot as the observed occurrence of this disease has been very low. The variety has reduced susceptibility to fungus and heat stress, and retains good summer color (i.e., does not show significant yellowing in summer).

Winter color: ‘B12’ has good winter color, being one of the last Stenotaphrum secundatum to go dormant, but under severe repeated frost it will turn brown.

### COMPARISON TRIALS WITH OTHER VARIETIES

A series of comparative trials were carried out among ‘B12’, ‘Sir Walter’, ‘SS-100’, ‘ST-85’ (Australian Patent No. 643567; not patented in the United States) and ‘Shademaster’ (unpatented; a commonly-grown Australian variety).

The characteristics used to identify the most similar varieties of common knowledge to ‘B12’ were: plant characteristics, main color of the stolon, internode length, and leaf width and length. Based on these criteria, ‘Sir Walter’ and ‘SS-100’ were selected as the most similar comparators. For a broader comparison, although these varieties are readily distinguishable by stolon color alone, varieties ‘ST-85’ and ‘Shademaster’ were also included in some of the trials.

The results of the comparison trials are shown in Tables 1-8 below. In summary:

‘B12’ has a shorter internode length than ‘Sir Walter’, and a longer internode length than ‘SS-100’ and ‘ST-85’.

‘B12’ has a longer leaf than ‘ST-85’, ‘SS-100’, ‘Sir Walter’ and ‘Shademaster’.

‘B12’ has an internode color of brown with predominant yellow green, while ‘SS100’ has a green internode, ‘Sir Walter’ has an internode of dark purple with a little green, and ‘ST-85’ and ‘Shademaster’ have a dark purple internode color.

‘B12’ grows at a similar speed to ‘Sir Walter’, as determined by both stolon growth across the ground and upward leaf growth. Both grasses have similar mowing rates. ‘B12’ grows only modestly faster across the ground from stolons than does ‘SS100’, but grows significantly faster in the leaf, therefore requiring more mowing than ‘SS-100’. It is noted that ‘B12’ grows unusually fast for a fine leaf Stenotaphnim secundatum. ‘ST-85’ is a more typical fine leaf variety, which grows more slowly than ‘B12’ in the leaf and stolon.

With respect to leaf width, it was determined that ‘B12’ has a finer leaf than ‘SirWalter’, ‘Shademaster’, and ‘SS100’. Further, when ‘B12 is established and forms a dense mat, it has a more angled infolded leaf than ‘Sir Walter’ and ‘SS100’, which gives ‘B12’ the appearance of being even more fine leafed than these other varieties. The angled leaf of ‘B12’ enhances the fine leafed appearance and makes it visually appear to be much more than 1 mm finer than ‘SS-100’.

Under conditions of low fertilizer, the best visual appearance ratings were for ‘B12’ and ‘SS-100’, followed by ‘Shademaster’.

The poorest visual appearance rating was for ‘Sir Walter’ followed by‘ST-85’.

In general, the varieties with highest visual ratings under low fertilizer conditions also had the highest scores for uniformity, density and greenness.

The comparison trials and results are described in more detail below.

### Comparison Trial 1: Internode Color, Internode Length and Leaf Length

A comparative trial was conducted in Clarendon, New South Wales, Australia in Spring/Summer 2002. ‘B12’, ‘Sir Walter’, ‘Shademaster’, ‘SS100’ and ‘ST-85’ were compared for stolon color (i.e., internode color), leaf length and internode length. Individual stolons were taken from open beds, and were then planted in 140 mm pots filled with soiless potting mix. Nutrition was maintained with slow release fertilizers, which were added at the time the Stenotaphrum were potted. The plants were grown in full sun in the open, with irrigation. The pots were seven months old at the time of trial. The plants did not flower during the trial. The trial design consisted of thirty pots of each variety arranged in a completely randomized design. Measurements were taken in December 2002. The results are shown in Table 1 below.

TABLE 1

| | B12 | Sir Walter | Shademaster | SS100 | ST-85 |
|---|---|---|---|---|---|
| Internode Color | Brown with | Dark purple | Dark purple | Green | Dark |
| (overall | predominant yellow- | with little green | | | Purple |
| appearance) | green | | | | |

Leaf Length (mm) - 4^{th} node from tip

| | | | | | |
|---|---|---|---|---|---|
| mean | 20.2 | 17.2 | 15.2 | 14.5 | 10.7 |
| standard deviation | 4.2 | 1.5 | 1.7 | 2 | 1.7 |
| LSD/sig | 3.13 | Ns | P ≦ 0.01 | P ≦ 0.01 | P ≦ 0.01 |

Average Internode Length (mm) - internodes 4 to 6

| | | | | | |
|---|---|---|---|---|---|
| mean | 50.4 | 57.1 | 52.3 | 44.1 | 34.85 |
| standard deviation | 4.6 | 4.8 | 2.9 | 3.1 | 2.9 |
| LSD/sig | 4.76 | P ≦ 0.01 | ns | P ≦ 0.01 | P ≦ 0.01 |

### Comparis n Trial 2: Leaf Width and Internode L ngth

Another comparison trial was conducted in Clarendon, New South Wales, Australia in October 2002, wherein three one-foot square pieces of sod of ‘B12’, ‘Sir Walter’ and ‘SS-100’ were compared with respect to internode length and leaf width. The sod was from Comparison Trial 3 below, and was planted in February 2002. The ten-month-old sod was grown using standard practices in the sod industry, and was harvested with a spade. Seventy-eight random samples were taken, 26 from each piece of sod, and were examined for internode length and leaf width. The results are shown in Table 2. Note that results for pot-grown samples will differ from turf-grown samples. For example, when the turf starts to mat in the pot, its growth is constrained, which affects some measurements. In addition, mowing turf-grown samples will affect some measurements. The pot trials, however, are useful for showing trends, and the observed trends were consistent throughout the trials.

TABLE 2

| | | |
|---|---|---|
| B12 | SIR WALTER | SS-100 |

Leaf width (mm) - 4^{th} node from tip

| | | | |
|---|---|---|---|
| Mean | 5.7 | 6.633 | 6.3 |
| Std deviation | 1.2 | 1.1 | 1.1 |
| LSD/sig | 0.7 | P ≦ 0.01 | P ≦ 0.01 |

Average internode length

| | | | |
|---|---|---|---|
| Mean | 30.04 | 39.87 | 25.23 |
| Std deviation | 15 | 16.3 | 12.4 |
| LSD/sig | 9.1 | P ≦ 0.01 | ns |

### Comparison Trial 3: Time to Harvest

In a third study, carried out in February 2002, approximately 15 square meters of bare ground in a plot in Clarendon, New South Wales, Australia was planted with stolons of‘B12’, ‘Sir Walter’, ‘SS100’ and ‘ST-85’. These plots were mown, watered and fertilized regularly, and were monitored for 12 months. At 9 months, ‘B12’ and ‘Sir Walter’ were ready to harvest. At 9 months and 2 weeks, ‘SS100’ was ready to harvest. Both ‘Sir Walter’ and ‘SS-100’ are considered fast-growing varieties of Stenotaphnim secundatum. In the same trial, ‘ST-85’ was not ready for harvest for another 2 months and 2 weeks (i.e., at 12 months). The results of this comparison are shown below in Table 3. Readiness for harvest was judged by ability to harvest the turf with a hand turf cutter.

TABLE 3

| Variety | Time from Planting to Harvest |
|---|---|
| B12 | 9 Months |
| Sir Walter | 9 Months |
| SS-100 | 9 Months 2 weeks |
| ST-85 | 12 Months |

### Comparison Trial 4: Leaf Width and Angle Comparison

In July 2003, a fourth trial comparing leaf width and leaf angle was conducted with ‘Sir Walter’ and ‘SS-100’, the two varieties of Stenotaphrum secundatum that were determined to be the most similar to ‘B12’. Upon inspection of mature, never-harvested material from Comparative Trials 2 and 3 above, it was observed that ‘B12’ had a finer leaf appearance than the leaf measurements in the first trials suggested. For this reason, a second comparison of leaf measurements was conducted. The results are shown in Table 4.

TABLE 4

| | | |
|---|---|---|
| ‘B12’ | SIR WALTER | SS-100 |

Leaf width (mm) - 4^{th} node from tip

| | | | |
|---|---|---|---|
| Mean | 4.85 | 6.02 | 5.88 |
| Std deviation | 0.8 | 0.9 | 0.7 |
| LSD/sig | 0.48 | P ≦ 0.01 | P ≦ 0.01 |

As expected, the comparison demonstrated that the leaf of ‘B12’ was finer than the leaves of the other two varieties. Nonetheless, it was surprising that the measurements indicated that ‘B12’ was only about 1 mm finer in leaf blade width. Based on visual appraisal of the varieties, ‘B12’ would appear to be at least 2 mm to 2.5 mm finer. Upon closer inspection and evaluation, it was noted that the leaf of ‘B12’ was far more infolded than the leaf of ‘Sir Walter’ and ‘SS100’. A system of measuring was devised by bending small pieces of wire at 40 degrees, 80 degrees, 120 degrees, and 150 degrees. A series of measurements was taken from each grass. All samples had leaf angles that fell below 180 degrees. Any sample that could not be clearly measured (e.g., borderline cases) was discarded. In total, six samples were discarded: three from ‘SS-100’, two from ‘Sir Walter’ and one from ‘B12’.

The results of this study can be seen in Table 5 below. It was found that ‘B12’ was more tightly infolded than the other varieties, as more of its leaves were folded at shaper angles, and less were folded at open angles. The somewhat finer leaf of ‘B12’, in combination with the more acute infold angle of the leaf, results in ‘B12’ having a much finer appearance than ‘Sir Walter’ and ‘SS-100’.

TABLE 5

| | 0 to 40° | 41 to 80° | | 121 to | 151 to |
|---|---|---|---|---|---|
| | Extremely | Highly | 81 to 120° | 150° | 180° |
| Angle | angled | angled | Angled | Open | Very open |
| Category | 5 | 4 | 3 | 2 | 1 |
| Frequency | 6 | 16 | 13 | 4 | 1 |
| of angle | | | | | |
| for ‘B12’ | | | | | |
| Frequency | 1 | 8 | 12 | 12 | 7 |
| of angle | | | | | |
| for Sir | | | | | |
| Walter | | | | | |
| Frequency | 3 | 10 | 16 | 8 | 3 |
| of angle | | | | | |
| for | | | | | |
| SS-100 | | | | | |

### Comparison Trials und r Low F rtilizer Conditions

Stenotaphrum varieties held at Kincumber, New South Wales, Australia since December 2002 were rated for performance on Mar. 21, 2003. The turf was in 140 mm pots and was transferred to Kincumber from the plants used in Comparison Trial 1, described above. The turf had not received fertilizer and was minimally watered. The plants were cut back (to pot edge) approximately four weeks prior to evaluation. Each variety was assessed for the indices shown in Table 6.

TABLE 6

| | | Visual | | No. | Leaf | | |
|---|---|---|---|---|---|---|---|
| Variety | Reps | rating | Uniformity | flowering | length | Density | Greenness |
| B12 | 4 | 8-9 | 8-9 | 25% | long | 8-9 | light |
| | | | | | | | green |
| | | | | | | | (RHS |
| | | | | | | | 144A) |
| Shademaster | 4 | 7-8 | 8-9 | 0% | med- | 8 | light |
| | | | | | long | | green |
| | | | | | | | (RHS |
| | | | | | | | 144A) |
| SS-100 | 4 | 8-9 | 9 | 0% | med- | 9 | medium |
| | | | | | long | | green |
| | | | | | | | (RHS |
| | | | | | | | 146A) |
| Sir Walter | 4 | 5-6 | 5 | 25% | med- | 5-6 | yellow |
| | | | | | long | | green |
| | | | | | | | (RHS |
| | | | | | | | 144A-B) |
| ST 85 | 2 | 5-6 | 8 | 100% | short | 8 | light |
| | | | | | | | green |
| | | | | | | | (RHS |
| | | | | | | | 144B) |

Performance Ratings:
Visual rating: 0 = dead, 10 = vigorous habit
Uniformity: 0 = poor, 10 = perfectly even over all replicates
Density: 0 = very sparse, 10 = very dense
Greenness: yellow green (chlorotic, nutrient deficient), light green, medium green, dark green (no nutrition deficiencies apparent).

The remaining trial stock at Clarendon, New South Wales, Australia from Comparison Trial 1 was evaluated in the same way on Mar. 26, 2003. These plants were adequately watered, but were not fertilized or pruned. Results are shown in Table 7.

TABLE 7

| | | Visual | | No. | Leaf | | |
|---|---|---|---|---|---|---|---|
| Variety | Reps | rating | Uniformity | flowering | length | Density | Greenness |
| B12 | 20 | 9 | 9 | 100% | long | 8-9 | light |
| | | | | | | | green |
| | | | | | | | (RHS |
| | | | | | | | 144A) |
| Shademaster | 20 | 8 | 8 | 25% | med | 8 | light |
| | | | | | | | green |
| | | | | | | | (RHS |
| | | | | | | | 144A) |
| SS-100 | 20 | 9 | 9 | 100% | med- | 8 | medium |
| | | | | | long | | green |
| | | | | | | | (RHS |
| | | | | | | | 146A) |
| Sir Walter | 20 | 5-6 | 5-6 | 50% | med- | 5-6 | yellow |
| | | | | | long | | green |
| | | | | | | | (RHS |
| | | | | | | | 144A-B) |
| ST-85 | 20 | 6-7 | 9 | 100% | short | 8-9 | Light |
| | | | | | | | green |
| | | | | | | | (RHS |
| | | | | | | | 144B) |

Performance Ratings:
Visual rating: 0 = dead, 10 = vigorous habit
Uniformity: 0 = poor, 10 = perfectly even over all replicates
Density: 0 = very sparse, 10 = very dense
Greenness: yellow green (chlorotic, nutrient deficient), light green, medium green, dark green (no nutrition deficiencies apparent).

The Kincumber stock (from the study shown in Table 6) was then harvested to determine average shoot yield as assessed by measuring the fresh weight of all above ground parts (leaves and stolons). Results are shown in Table 8.

TABLE 8

| | Mean Shoot Fresh |
|---|---|
| Variety | Weight (g) |
| B12 | 80 |
| Shademaster | 71 |
| SS-100 | 105 |
| Sir Walter | 39 |
| ST 85 | 55 |

### Conclusions of Low Fertilizer Trials

The best visual appearance ratings were for ‘B12’ and ‘SS-100’, followed by ‘Shademaster’. The poorest visual appearance rating was for ‘Sir Walter’ followed by ‘ST-85’.

In general, the varieties with highest visual ratings also had the highest scores for uniformity, density and greenness. In other words, these varieties have complete ground coverage, even growth and a greener appearance than the poorer varieties.

Since the plants in these studies were not mown, it appears that longer leaf length combined with uniform growth generally contribute to a positive visual assessment. Leaf length correlated with shoot mass for the longer leaf varieties ‘B12’ and ‘SS-100’.

### Comparative DNA Analysis of ‘B12’ with other Turfgrasses

Randomly Amplified Polymorphic DNA (RAPD) analysis of ‘B12’ in comparison with other turfgrasses was performed using a series of ten-mer primers from Operon Technologies, Inc. (Alameda, Calif.) as described below:

Plant Material

Samples from six different turfgrasses were provided by Todd Bunnell, (Clemson University). The turfgrass varieties used in the analysis were: ‘Bitterblue’, ‘Palmetto’™, Woemer Classic’, ‘Raleigh St. Augustine’, ‘Floratam’, and ‘B12’.

DNA Isolation

DNA was isolated from the leaf blades using the DNeasy procedure from Qiagen (Valencia, Calif.). The DNA extracts were quantified so that equal amounts of DNA could be used in the amplifications.

Amplification Primers

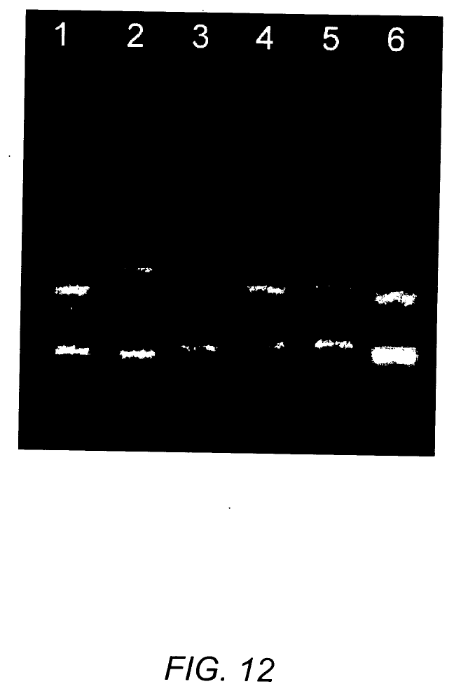

One hundred ten-mer primers from Operon Technologies, Inc. (Alameda, Calif.) were used in the comparisons among the six turf grass samples. Of these, 32 were used to evaluate the complete set of samples (OPB 1, OPB 6, OPB 7, OPB 11, OPB 12, OPB 15, OPB 17, OPB 18, OPB 19, OPC 4, OPC6, OPC 8, OPC 9, OPC 10, OPC 11, OPC 12, OPJ 7, OPJ 9, OPJ 10, OPJ 11, OPJ 13, OPK 1, OPK4, OPK 10, OPK 11, OPK 15, OPAC 2, OPAC 3, OPAC 10, OPAC 11, OPAC 18, OPAC 19, OPAC 20) FIG. 12 shows the results when the primer OPC4 (CCGCATCTAC; SEQ ID NO:1) was used.

Amplification

The PCR reaction was carried out using TaKaRa Taq polymerase (Takara Bio Inc.) in the manufacturer's supplied buffer with a final concentration of MgCl₂ of 2.5 mM. Each reaction contained 25 μg of leaf DNA. The PCR reactions were subjected to a hot start with the buffer heated to 85° C. before the addition of Taq polymerase. All the reactions were carried out using MJR PTC-100 thermal cyclers (MJ Research, Inc.).

The amplification program consisted of:

1. 1 min 96° C.

2. 1 min 94° C.

3. 1 min 35° C.

4. 2 min 72° C.

5. cycle to step 2 45 times

6. hold at 72° C. for 10 minutes

7. cool to 4° C.

8. Hold

Gel Electrophoresis and Photography

The PCR products were separated on a 1.5% agarose gel in Tris-borate-EDTA containing ethidium bromide and visualized using a Bio-Rad transilluminator. Images were captured with a Kodak DC290 camera. Lane 1=‘Bitterblue’; Lane 2=‘Palmetto’™; Lane 3=‘Woerner Classic’; Lane 4=‘Raleigh St. Augustine’; Lane 5=‘Floratam’; Lane 6=‘B12’.

RAPD Analysis

Randomly Amplified Polymorphic DNA (RAPD) analysis of the six turfgrass samples was carried out as described above. All of the samples could be distinguished from each other using one or more of the ten-mer primers. The results with the OPC4 primer are shown in FIG. 12. As shown in the figure, ‘B12’ can be distinguished by RAPD analysis from the other turfgrasses evaluated using the OPC4 primer.

That which is claimed is:

1. A new and distinct variety of Stenotaphrum secundatum named ‘B12’, substantially as illustrated and described herein.


SEQUENCE LISTING (1 sequence)

SEQ ID NO: 1 - DNA, 10 bp, Artificial
  Primer
ccgcatctac